(19) United States
(12) Patent Application Publication
(10) Pub. No.: US 2013/0243829 A1
(43) Pub. Date: Sep. 19, 2013

(54) ENHANCEMENT OF IMMUNE RESPONSE BY TRANSFER FACTOR

(71) Applicant: Joseph C. Ramaekers, Aptos, CA (US)

(72) Inventor: Joseph C. Ramaekers, Aptos, CA (US)

(21) Appl. No.: 13/877,309

(22) PCT Filed: Sep. 30, 2011
(86) PCT No.: PCT/US11/54268

§ 371 Date: Jun. 3, 2013

Related U.S. Application Data
(60) Provisional application No. 61/389,190, filed on Oct. 1, 2010.
(60) Provisional application No. 61/389,230, filed on Oct. 2, 2010.

(51) Int. Cl. A61K 38/19

(52) U.S. Cl. CPC A61K 38/19
USPC 424/400; 424/278.1

(57) ABSTRACT

In certain aspects, methods and kits are provided for use in enhancement of immune response by administering to a subject an effective amount of a composition containing at least one transfer factor.

### CROSS-REFERENCES TO RELATED APPLICATIONS

This application claims benefit under 35 U.S.C. §119(e) of U.S. Provisional Application No. 61/389,190, filed Oct. 1, 2010 and U.S. Provisional Application No. 61/389,230 filed Oct. 2, 2010, and for the United States is a continuation-in-part of U.S. application Ser. No. 12/631,745, filed Dec. 4, 2009. U.S. application Ser. No. 12/631,745, in turn, is a continuation of U.S. application Ser. No. 11/492,464, filed Jul. 24, 2006, which claims benefit under 35 U.S.C. §119(e) of U.S. Provisional Application No. 60/701,860, filed Jul. 22, 2005. U.S. application Ser. No. 11/492,464, in turn, is a continuation-in-part of U.S. application Ser. No. 11/106,054, filed Apr. 13, 2005, which claims benefit under 35 U.S.C. §119(e) of U.S. Provisional Application No. 60/573,113, filed May 20, 2004, and U.S. Provisional Application No. 60/649,363, filed Feb. 1, 2005. U.S. application Ser. No. 12/631,745 is also a continuation of U.S. application Ser. No. 11/237,316, filed Sep. 27, 2005, which is a division of U.S. application Ser. No. 10/136,854, filed Apr. 30, 2002, now U.S. Pat. No. 6,962,718, which is a continuation-in-part of U.S. application Ser. No. 09/847,036, filed Apr. 30, 2001, now U.S. Pat. No. 6,506,413. The disclosures of each of the foregoing patents and patent applications are hereby incorporated by reference herein in their entireties.

### BACKGROUND OF THE INVENTION

Transfer factors, which are produced by leucocytes and lymphocytes, are small water soluble polypeptides of between about 44 amino acids that stimulate or transfer cell mediated immunity from one individual to another and across species but do not create an allergic response. Since transfer factors are smaller than antibodies, they do not transfer antibody mediated responses and they do not induce antibody production. The properties, characteristics and processes for obtaining transfer factor or transfer factors are discussed in U.S. Pat. Nos. 4,816,563; 5,080,895; 5,840,700, 5,883,224 and 6,468,534, the contents of which are hereby incorporated by reference into the present application.

Transfer factor has been described as an effective therapeutic for Herpes simplex virus (Viza, et al.), a treatment for acne blemishes, U.S. Pat. No. 4,435,384 and as a treatment against C. albicans (Khan et al.). Transfer factor has also been used to treat intestinal cryptosporidiosis in recipients treated with specific transfer factor (McMeeking, et al.). Still, et al. also showed that chicken pox infections were prevented by pretreatment of children treated with transfer factor from individuals that had chicken pox or who in other words had been sensitized to the varicella antigen. The antigen specific transfer factors are the most well studied and have been demonstrated to be able to convey the antigen recognition ability of the experienced donor to the naive recipient. It may be assumed that the individual or animal that is the source of the transfer factor has been sensitized to the antigen of interest. However, transfer factor as found in commercial bovine colostrum extract coming from a pool of animals (e.g., cows) contains the acquired immunity from all of the pool and therefore provides a type of generalized adoptive transfer of immunity. Transfer factors or transfer factor can be obtained from a dialyzable extract of the lyzed cells or from an extract of extracellular fluid containing transfer factor. Common sources of transfer factors are colostrums and ova. It is common practice to refer to preparations that contain transfer factor by the name of the active component (i.e., transfer factor or TF). Transfer factor extract containing transfer factors is also herein referred to as transfer factor. Transfer factor from bovine colostrum extract is defined as defatted water soluble material from colostrum that will pass through a nominal 10,000 molecular weight filter. The colostral derived transfer factor has been prepared with activity against various organisms including infectious bovine rhinotracheitis virus. One of the specific effects of transfer factor is a significantly increased natural killer (NK) cell activity. Natural killer cells provide protection against viruses as part of the innate immune defense system.

Although transfer factor is a polypeptide, it has been reported that it is surprising stable in the gastrointestinal tract. For example, Kirkpatrick compared oral versus parental administration of transfer factor in clinical studies. Kirkpatrick, Biotherapy, 9:13-16, 1996. He concluded that the results refute any arguments that the acidic or enzymatic environment of the gastrointestinal tract would prevent oral therapy using transfer factors.

When attempts were made to sequence TF, it was reported that an N-terminal end of the transfer factor peptide is resistant to sequential Edman degradation. Kirkpatrick, Molecular Medicine, 6(4):332-341 (2000). Kirkpatrick's work on the structural elucidation of transfer factor's demonstrated failure of the peptide to sequentially fragment under standard Edman degradation conditions indicated the presence of some sort of blocking moiety on the terminal amine group Kirkpatrick Molecular Medicine, 6(4):332-341 (2000).

In view of this, one skilled in the art would expect that a peptide that is stable to Edman degradation would also be stable toward enzymatic degradation that occurs in the rumen. However, it has since been shown that transfer factor is not as stable as once believed. It appears to be particularly unstable in the digestive tract of ruminants.

Transfer factors have been used successfully in compositions for treating animal diseases and syndromes, including those in ruminants. See, for example U.S. Pat. No. 6,962,718.

Immunity is the state of being protected from a disease. It can be achieved by passive or active immunization. Passive immunization is the transfer of active humoral immunity in the form of antibodies or immune cells, from one individual to another. Passive immunization can occur naturally, as when maternal antibodies are transferred to the fetus through the placenta, when maternal antibodies are transferred to the neonate through colostrum, or artificially, as when high levels of antibodies specific for a pathogen or toxin are transferred to an individual requiring immunity. While transfer factor has been proven to enhance cellular immunity, studies have demonstrated that it does not influence the level of immunoglobulin in any significant way. Bystroñ, J. et al., Na\check{s}e Zku\check{s}enosti S Lé\check{c}bou Recidivujicich Herpetických Infekci Kombinovanou Nespecifickou Immunostimilaci, Casopis Lekaru Ceskych, 131:5, 137-141, Mar. 13, 1992.

Active immunization entails the education of a host's own immune cells to react against a molecule or target, typically carried on a foreign molecule and introduced into the body. Active immunization can occur naturally, as when a subject comes in contact with, for example, a microbe, and the subject becomes immunized against the microbe. Artificial active immunization may be induced when the microbe, or parts of it, is administered to a subject. Vaccination is an active form of immunization.

### BRIEF SUMMARY OF THE INVENTION

In certain aspects, the invention relates to methods of improving a vaccine-induced immune response in a subject comprising administering to a subject in need thereof an effective amount of a composition and/or formulation comprising at least one transfer factor.

In other aspects, the invention relates to kits comprising at least a first container comprising a composition and/or formulation comprising at least one transfer factor.

### DETAILED DESCRIPTION OF THE INVENTION

The present invention relates, in certain aspects, to methods of altering antigen-induced immune response following vaccination of animals, including humans, using compositions comprising transfer factor. Other U.S. patents and U.S. patent applications relate to the present invention, including without limitation, U.S. Pat. Nos. 6,506,413 and 6,962,718, U.S. Patent Provisional Application Nos. 60/573,113, 60/649,363, 60/701,860, and 60/814,777, U.S. patent application Ser. No. 11/762,727, U.S. Patent Application Publication Nos. 2006/0029585 A1, 2006/0073197 A1, and 2007/0128253 A1, all of which are incorporated herein by reference. Also related are PCT publications WO/2002/087599 and WO/2005/112891, incorporated herein by reference.

In certain aspects, the invention relates generally to the methods of use of compositions comprising transfer factor in providing health benefits to humans and animals; such benefits may include, without limitation, enhancing immune response following vaccination, decreasing the decay of immune response over time following vaccination, and prolonging the interval of re-vaccination.

Recognizing the difficulty in achieving adequate antibody response following vaccination of humans and animals, and the added physiologic and economic benefit that would accompany a decrease in the required number of initial vaccinations and an increase in the time between annual boosters, methods were investigated for improving immune performance of vaccinated individuals and herds. These studies disclosed a previously unknown response to antibody production and longevity following administration of transfer factor alone and in combination with glucans.

In certain aspects, the present invention is directed to methods for enhancing a vaccine-induced immune response in a subject comprising the administration to a subject of an effective amount of a composition and/or formulation comprising at least one transfer factor.

It has been found by the present inventor that the administration of compositions comprising transfer factor provides surprising and unexpected improvements in enhancing protective immunity induced by vaccination.

In certain embodiments, the present invention is directed to methods for enhancing the immune response elicited by administration of a vaccine to a subject. In preferred embodiments, these methods comprise the administration to subject of an effective amount of a composition and/or formulation comprising at least one transfer factor.

As used herein, the term “effective amount” refers to an amount of a compound, material, composition, formulation and/or dosage form as described herein that may be effective to achieve a particular biological result. Such results may include, but are not limited to, enhancement of an immune response induced by administration of a vaccine.

Transfer Factor

According to certain aspects of the invention, compositions and formulations are provided comprising transfer factor. According to certain embodiments of the invention, various forms of transfer factor may be used. They include, without limitation, excreted transfer factor released from transfer factor containing cells such as lymphocytes, leukocytes, and ova, and collected from extracellular fluids such as colostrums and blood. Another form includes preexcreted transfer factor found within the cell or on the cell surface. In certain embodiments of the invention, substantially purified transfer factor originating from leukocytes, colostrum, or ova and having a molecular weight of less than 10,000 daltons and a specific activity of at least 5000 units per absorbance unit at 214 nanometers, may also be used. The transfer factor used in the Examples of this invention and referred to in the following Tables and further referred to in the rest of the detailed description is generally extracted from colostrum collected from a general pool of lactating cows; although, in some cases, it is derived from eggs. Though bovine colostral derived transfer factor was generally used to develop the formulations of this invention, it is well known to anyone skilled in the art that other kinds and sources of transfer factor could be used.

Alternative sources of transfer factor include, but are not limited to, avian transfer factor, ova transfer factor, and transfer factor isolated from colostrum collected from non-bovine animals such as goats, pigs, horses and humans. In addition, combinations of transfer factors from any number of sources may be used in the formulations of the instant invention. Transfer factor may also be derived from recombinant cells that are genetically engineered to express one or more transfer factors or by clonal expansion of leukocytes.

Isolation of Transfer Factor

In certain embodiments of the invention, transfer factor may be obtained from colostrum. In a preferred embodiment, transfer factor is obtained from bovine colostrum. The fraction of colostrum comprising material having a molecular weight of approximately 10,000 daltons (Da) and below is designated as transfer factor. A fraction obtained that is approximately 10,000 to approximately 150,000 Da is designated an antibody fraction, also known as an antibody-colostrum fraction. In certain embodiments, a colostral fraction having a molecular weight of about 10,000 to about 400,000 Da may be used as an antibody fraction. The fraction comprising material having a molecular weight of approximately 10,000 Da and above is designated as the growth factor fraction. The growth factor fraction may include high molecular weight proteins.

In certain embodiments, an antibody fraction comprises antibodies from about 1% to about 99%, about 5% to about 95%, about 10% to about 90%, about 15% to about 85%, about 20% to about 80%, about 25% to about 75%, about 30% to about 70%, about 35% to about 65%, about 40% to about 60%, about 45% to about 55%, or about 50% by weight, the remainder comprising other colostrum components.

According to certain embodiments of the invention, transfer factor, as used in formulations described herein, particularly when not defined as obtained from an avian source, may be further defined as defatted water soluble material from bovine colostrum that will pass through a nominal 10,000 molecular weight filter.

In other embodiments, the transfer factor may be obtained from an avian source. In one embodiment, chickens are given a feed mixture containing excrement from an animal, including without limitation, at least one selected from the group consisting of a human, a fish, a goat, a llama, an alpaca, a pig, a sheep, a cow, and a horse. The excrement will contain a large variety of pathogens and upon administration in a feed to an animal, the animal will develop transfer factor and/or antibodies to such pathogens. Avian transfer factor can then be obtained from the eggs produced by the above-treated chickens. In certain embodiments, transfer factor may be found in whole egg yolks.

Alternative kinds of transfer factor include, but are not limited to, targeted transfer factors. Target transfer factors include transfer factor collected from sources which have been exposed to (1) one or more viral or otherwise infectious organisms; (2) one or more antigens that produce an immune response; or (3) a combination of organisms and antigens. The term antigen is defined herein is anything that will initiate the cell mediated immune response. Examples of such viral or other infectious organisms include Herpes Simplex Virus 1, Herpes Simplex Virus 2, H. Pylori, Camphobacter and Chlamydia, Bovine Rhinotracheitis Virus, Parainfluenza, Respiratory Syncytial Virus Vaccine, modified live virus, Campylobacter Fetus, Leptospira Canicola, Grippotyphosa, Hardjo, Leterohaemorrhagiae, Pomona Bacterin, Bovine Rota-Coronavirus, Escherichia Coli Bacterin, Clostridium Chauvoei, Septicum, Haemolyticum, Novy, Sordellii, Perfringens Types C & D, Bacterin, Toxoid, Haemophilus Somnus, Pasteurella Haemolytica, Multocida Bacterin. However, one of skill in the art would readily recognize that a wide variety of other viral and otherwise infectious organisms can find use in the instant invention.

Additionally, transfer factor and antibodies may be derived from any suitable source, as described, for example, in U.S. Pat. Nos. 4,816,563; 5,080,895; 5,840,700; 5,883,224; and 6,468,534; and U.S. patent application Ser. No. 11/762,727, the contents of which are hereby incorporated by reference herein.

In certain embodiments, the component of a given formulation that is referred to as the “transfer factor” may optionally include a colostral component of higher molecular weight; for example, a portion of the fraction referred to above as an antibody or antibody-colostrum fraction. In certain embodiments, the mammalian “transfer factor” component of a formulation comprises both transfer factor fraction and antibody fraction. In certain preferred embodiments, “mammalian transfer factor” comprises about 70% transfer factor fraction from colostrum (i.e., 10,000 Da or below colostrum fraction) and about 30% antibody colostrum fraction. In other preferred embodiments, “mammalian transfer factor” comprises about 80% transfer factor fraction from colostrum and about 20% antibody colostrum fraction. (The foregoing are in weight percents of the composition). In certain embodiments, the “transfer factor” component of the composition or formulation may include one or both of mammalian and avian transfer factor.

Lyophilization

The present invention also provides compositions and formulations that have one or more lyophilized component(s). Lyophilization or “freeze-drying” is a process well known to those of ordinary skill in the art. For example, some techniques of lyophilization are described in Akers, Michael J., Chapter 41 in Remington The Science and Practice of Pharmacy, 828 (David B. Troy ed., Lippincott Williams & Wilkins 2006), which is incorporated herein by reference. In certain embodiments, formulations and/or compositions of the present invention may include lyophilized transfer factor. In certain embodiments, transfer factor, which may be lyophilized, may be combined with one or more of: an antibody or an antibody fraction, a growth factor fraction, or some other colostral fraction; one or more of which may, in certain embodiments, be lyophilized. In other embodiments, additional components of formulations and compositions of the invention may be lyophilized, including, without limitation, other peptides and proteins.

In other embodiments of the invention, transfer factor and/or additional components of formulations and compositions described herein may be processed by suitable methods other than lyophilization. Such methods include, but are not limited to, spray drying.

Formulations

In certain embodiments of the invention, transfer factor may be provided in a formulation that further comprises one or more additional ingredients. In certain preferred embodiments, the formulation comprises transfer factor and at least one glucan. In some embodiments, transfer factor fraction and at least one glucan is provided in a formulation that further comprises one or more additional ingredients. In certain embodiments, the transfer factor fraction may be lyophilized. In certain embodiments, the optional growth factor colostral fraction, other colostral fraction, antibody or antibody fraction may be lyophilized.

In a preferred embodiment, transfer factor is present in the formulation in the amount of about 10 mg to about 12 gm/oz, more preferably about 100 mg to about 6 gm/oz and most preferably about 10 mg to about 3 gm/oz. In certain preferred embodiments, such a formulation comprising transfer factor is provided to an animal in an amount of about 1 oz per 1000 lb of animal.

In certain embodiments of the invention, formulations are provided which comprise glucans. Glucans may be derived from any suitable source, including, but not limited to, fungi, oats, and yeast. Preferably, glucans are present in or derived from fungi. In certain embodiments, the glucans which may be included in the formulations are present in whole fungi. In certain preferred embodiments, glucans are present in or derived from Cordyceps, more preferably, Cordyceps sinensis.

In certain embodiments, glucans are derived from hybrid strains of fungi. In a preferred embodiment the hybrid glucans used in the invention are present in, or derived from, hybrid strains of Cordyceps and in particular Cordyceps sinensis. One technique to induce the hybridization of Cordyceps involves plating two different strains or species on a single agar plate which has been inoculated with rattlesnake venom as described in, for example, U.S. Patent Application Publication No. 2006/0073197, published Apr. 6, 2006, and U.S. Patent Application Publication No. 2007/0128253, published Jun. 7, 2007, each of which is incorporated herein by reference. In a preferred embodiment, the hybrid strain producing the hybrid glucans that may be used in compositions and formulations of the invention is Cordyceps sinensis Alohaensis, which is available from Pacific Myco Products, Santa Cruz, Calif.

In addition to Cordycep sinensis hybrids, suitable sources of glucans may include, but are not limited to, Agaricus blazeii, Coriolus, Poira Cocos, Inonotus obliquus, Maitake Mushroom, Shiitake Mushroom, and combinations thereof.

When glucans are used, the formulation preferably contains about 10 mg to about 18 gm of whole organism/oz, more preferably about 100 mg to about 10 gm of whole organism/oz and most preferably about 100 mg to about 5 gm of whole organism/oz.

Equivalent amounts of purified or partially purified glucan as well as the nucleosides associated therewith (e.g., Cordycepin (3′deoxyadenosine), adenosine and N6-(2 hydroxyethyl)-adenosine) may also be used.

In certain preferred embodiments of the invention, formulations that may be administered to a subject, may comprise, in addition to transfer factor, which, in preferred form, may be derived from mammalian and/or avian source, one or more of the following: at least one glucan, mammalian antibody-colostrum fraction, mammalian growth factor fraction, other mammalian colostral fraction, and avian antibodies or antibody fraction (which may, in certain embodiments, be obtained from whole egg yolk).

In certain preferred embodiments, compositions may further comprise one or more of inositol hexaphosphate (Ip6), mannans, olive leaf extract, and phytosterols. In certain preferred embodiments, mannans are derived from Aloe vera. In certain preferred embodiments, phytosterols may be derived from soya bean.

In certain embodiments, compositions may further comprise one or more of lactic acid producing bacteria, ascorbic acid, Vitamin A, Vitamin D, Vitamin E, Vitamin B 1, Vitamin B2, Vitamin B12, dipotassium phosphate, potassium chloride, magnesium salts or chelates, and calcium pantothenate.

In certain embodiments, compositions and formulations comprising transfer factor may be combined with minerals, antioxidants, amino acids, and other nutraceuticals.

Encapsulation

In certain embodiments, the invention provides compositions and formulations in which one or more components are encapsulated. Encapsulation may be achieved by mixing the component to be encapsulated with a hydrophobic substance or a lipid to form a coating around the component. Encapsulation may protect labile components from inactivation in the gastrointestinal tract. Such encapsulation may be important especially in the case of ruminants where digestion within the rumen has been found to interfere with the administration of certain labile factors. Enhanced bioavailability has been demonstrated, for example, when a transfer factor is encapsulated and administered to ruminants.

Previous use of non-encapsulated transfer factor in ruminants, e.g., cows, produced significant beneficial results. See, e.g. U.S. Patent Publication 2003/0077254, published Apr. 24, 2003 incorporated herein by reference in its entirety. Subsequently, it was discovered that transfer factor was not stable by oral administration in a stressed population of cattle. After discovering that transfer factor is inactivated in vitro in the presence of rumen fluid and flora, it was determined that prior success with transfer factor in ruminants was due to the presence of the esophageal groove. When not stressed, the esophageal groove provides partial bypass of the rumen. However, in a stressed population the esophageal groove closes and shunts the transfer factor formulation into the rumen. It was discovered that encapsulating transfer factor and/or glucans with a hydrophobic substance or a lipid to form an encapsulated formulation is sufficient to provide substantial by-pass of (e.g., about 85%) of the rumen even in a stressed population.

While not intending to be bound by any theory or theories of operation, it is believed that encapsulation of transfer factor fraction may increase its bioavailability upon administration to fermenting animals, such as adult ruminants.

In preferred embodiments, transfer factor is encapsulated by mixing with a hydrophobic substance or a lipid to form a coating around the transfer factor(s). In additional embodiments, one or more additional components including antibody, antibody fraction, growth factor fraction, other colostral fractions, and/or glucans may be encapsulated. Other optional components of compositions and formulations of the invention may be encapsulated, such as, without limitation, inositol hexaphosphate, olive leaf extract, mannans, phytosterol, vitamin C and mixtures thereof. The transfer factor, antibody or antibody fraction and/or additional optional components may each be individually encapsulated or encapsulated as a mixture. Alternatively, the entire formulation can be encapsulated. The encapsulated component(s) and/or formulation can be produced in a variety of ways. In a preferred embodiment, each of the transfer factor, glucans, antibody or antibody fraction and/or additional labile component(s) in the formulation may be encapsulated as described in U.S. Pat. Nos. 5,190,775, 6,013,286 and U.S. Application 2003/0129295, each of which is incorporated herein by reference in its entirety.

The transfer factor may be encapsulated with a hydrophobic or lipid coating that is preferably between about 25% and about 150 wt % of the transfer factor, about 50-150 wt % and about 75-125 wt %, with an equal weight being most preferred.

In additional embodiments of the invention, additional components may be used in the formulation administered. Particular components may be encapsulated. For example, IP6, beta-sitosterol, olive leaf extract, aloe extract matter and/or vitamin C may be used; in certain embodiments, one or more of these components may be encapsulated. In preferred embodiments, IP6 is present at between 10 mg and 3 gm/oz, or one preferably between 100 mg and 2 gm/oz, and most preferably between 100 mg and 1 gm/oz. The beta-sitosterol is preferable in the amount of between 10 mg and 3 gm/oz, or preferably between 100 mg and 2 gm/oz, and most preferably between 100 mg and 1 gm/oz. Olive leaf extract is preferably present in the amount of 2 mg to 2 gm/oz, more preferably between 5 mg and 1 gm/oz, and most preferably between 5 mg and 500 gm/oz. Aloe extract is preferably present at between 2 mg and 1000 mg, more preferably between 5 and 500 mg/oz, and most preferably between 5 and 250 mg/oz. Vitamin C may be present at between 10 mg/oz and 10 gm/oz, or preferably between 100 mg and 8 gm/oz, and most preferably between 100 mg and 5 gm/oz.

In certain embodiments, glucans of the formulation may be encapsulated, preferably with a hydrophobic or lipid coating. It is preferred that the amount of hydrophobic or lipid coating be between about 25% and 150 wt % of the glucan, about 50-150 wt %, or about 75-125 wt %, with an equal weight being most preferred.

Methods of Administration

In certain embodiments, the present invention provides methods of treating a subject by administering a composition or formulation comprising transfer factor, as described herein. In certain embodiments, such methods are provided for enhancing a vaccine-induced immune response in the subject by administration of a composition or formulation comprising at least one transfer factor. In particular embodiments, methods are provided of increasing antibody titer in an immunized subject by the administration of a composition or formulation comprising at least one transfer factor. In certain embodiments, the transfer factor may be selected from the group consisting of bovine colostrum transfer factor, avian transfer factor, and a combination thereof.

In certain embodiments, transfer factor compositions and/or formulations may be administered to a subject orally. In additionally embodiments, transfer factor compositions and/or formulations may be administered to a subject by other suitable means, including, but not limited to, subcutaneously. In certain embodiments, transfer factor compositions and/or formulations may be administered to a subject parenterally.

In certain embodiments, transfer factor compositions and/or formulations may be included in food. Preferred embodiments for human consumption include, but are not limited to incorporation of transfer factor formulations in processed foods such as cereals, snacks, chips, or bars. Preferred embodiments for animal consumption include, but are not limited to, transfer factor formulations admixed in feed pellets, salt licks, molasses licks or other processed feed products.

Other methods of administration to animals, including, but not limited to, drenching, may also be employed.

In certain embodiments, oral or subcutaneous administration of a composition comprising transfer factor may be achieved by the use of a time-release or controlled-release implantable dosage form. Examples of suitable implantable dosage forms have been described in U.S. Pat. Nos. 5,665,363, U.S. Pat. No. 6,290,980, and RE 39,014 (a reissue of U.S. Pat. No. 6,290,980).

Oral Time-Release Formulations Comprising Transfer Factor

In certain embodiments, the invention relates to formulations that provide controlled (delayed) release of active agents. In certain embodiments, these formulations may be administered orally. In certain embodiments, the formulation may comprise a combination of water soluble and water insoluble polymers. Preferably, the polymers are cellulose polymers. In certain embodiments, the formulation comprises a combination of a water soluble cellulose polymer, hydroxypropyl cellulose and a water insoluble ethylcellulose. Preferably, ethylcellulose is present in the formulation at greater than about 30% w/w. Ruminating animals have natural cellulase enzyme in the rumen. Monogastrics (humans, swine, dogs, cats) and pre-ruminating ruminants break this water soluble (ethylcellulose) down to an extent without cellulase present. While not intending to be bound by any theory, it is believed that this may be attributed channels forming in the solid pellets and/or slugs and the water solubility of the actives themselves. It is expected that release is delayed in monogastrics. Additional ingredients may function as binder, flow-aid during pelleting, and lubricant.

In certain embodiments, one or more active agents are blended then compressed together with a combination of water soluble and water insoluble polymers to create a matrix. Preferably, the polymers are water soluble hydroxypropyl cellulose and water insoluble ethylcellulose polymers. In certain embodiments, the formulation comprises one or more active agents coated with a time-release coating comprising a combination of water soluble and water insoluble polymers. Preferably, the polymers are water soluble hydroxypropyl cellulose and water insoluble ethylcellulose polymers.

In certain embodiments, the ingredients may be blended, then “slugged” on a tablet press. These slugs may be provided for inclusion in large gelatin capsules and delivered orally.

In another embodiment, the blends may have the powders compressed (densified) and fed en masse through a multiple orifice to make extruded strands that are cut with a blade to form small irregularly-shaped pellets.

In preferred embodiments, an active ingredient powder (comprising transfer factor and other active components as described herein) comprises about 50% of the solid dosage form leaving about 50% for functional excipients. In one embodiment, an exemplary formulation for oral administration comprises:

“Active” powder blend(s) (50.0% w/w)

Ethylcellulose—filler and controlled release polymer (39.5% w/w).

Hydroxypropyl cellulose (8.0% w/w)—binder and pore-former

Stearic acid (2.0% w/w)—pelleting lubricant

Fumed silica (0.5% w/w)—powder glidant

It is suggested that many excipients known in the art may be combined and used to achieve similar results.

In certain embodiments, a time-release formulation comprises a polymer-wrapped formulation comprising transfer factor. The formulation may further comprise additional components as described herein for administration in conjunction with transfer factor. In certain embodiments, components of the formulation may be lyophilized. In certain embodiments, components of the formulation may be encapsulated.

In certain embodiments, the active ingredients are being released over approximately 30 hours. This may be useful, for example, in eliminating the second day of administration which is necessary for treatment which requires administration on day one and two using current dosing protocols.

By giving a normal bolus with quick release and a bolus of, for example, about 30 hour release, day two of administration is eliminated, thus removing a huge barrier in labor and compliance in the administration of active ingredients, and minimizing stress-induced vaccine failure. In certain aspects, a method is provided requiring only one administration day one for the subject. In certain embodiments, the subject may include, but is not limited to, both adult cattle and baby calves.

Certain embodiments provide methods of administration of time-release oral formulations. In certain embodiments, a method of administration is effective for ruminant animals. In preferred embodiments, the ruminant animal is a bovine.

In certain aspects, methods are provided to bypass the rumen with degradable glucans and peptides, and release them over an extended time period orally. Thus, oral delivery of compositions comprising transfer factor that accomplishes both rumen bypass and time release may be achieved by formulations and methods provided herein. In a preferred embodiment, active ingredients are released over an approximately 30 hour time span.

In other embodiments, methods of time-release oral administration is provided for animals that are not ruminants. In preferred embodiments, the animal is a calf.

In one aspect, the components of the compositions and formulations of the present invention may have an effect upon administration individually, such as for example the enhancement of immune response, but upon administration in one or more combinations, have an effect that is synergistic. By “synergistic” is meant an enhancement of the effect of one or more combined components in a more than additive fashion relative to the effect of each component when used alone.

In certain embodiments, the present invention provides methods involving administration of compositions and/or formulations comprising transfer factor to a subject. As used herein, the term “subject” is used to mean an animal, including, without limitation, an avian or a mammal. Mammalian subjects include, without limitation, human and non-human primates, cattle, horses, sheep, swine, goats, deer, lama, dogs and cats.

In certain embodiments, the present invention provides methods for improving vaccine-induced immune response of the subject by administration of transfer factor. In particular embodiments, methods are provided for increasing the antibody response during and following vaccination. Antibody response is measured by determining the titer by laboratory testing. A preferred laboratory method is the rapid fluorescent focus inhibition test.

In certain aspects, methods according to the invention may be used to increase the duration of effective vaccine-induced immunity. This extends the time between initial vaccination and booster shots, while retaining effective protection against the targeted pathogen.

Preferably, the number of booster shots following initial vaccination of a subject against a specific pathogen is reduced to less than 4, less than 3, or less than 2. Preferably, the effective duration of antibody protection following vaccination is more than 1 year, more than 2 years or more than 3 years.

The number of subjects mounting an effective antibody response following administration of the invention before, during or after vaccination increases the vaccine efficacy rate by at least 5%, preferably at least 10%, and most preferably by at least 20%.

Kits

In one aspect, the present invention provides kits suitable for treatment of a subject, for example, an animal. In certain embodiments, a kit is provided for enhancement of vaccine response in a subject. The kits may further include instructions for use. Instructions may be included as a separate insert and/or as part of the packaging or container, such as a label affixed to a container or as a writing or other communication integrated as part of a container. The instructions may inform the user of methods of administration of the compositions and formulations contained therein, precautions, expected results, warnings concerning improper use, and the like.

In one embodiment, the kit includes a first container having a composition or a formulation that includes a transfer factor. In addition to at least one transfer factor, the formulations and compositions of the present invention may also include one or more of a glucan, a colostral fraction, such as a high-molecular weight or growth factor fraction, an antibody or antibody fraction.

In certain embodiments, a kit may comprise at least a first container comprising a composition comprising at least one transfer factor, wherein said composition is selected from the group consisting of encapsulated transfer factor, time-release polymer wrapped transfer factor, and a combination thereof.

In certain embodiments, a kit may comprise at least a first container comprising a composition comprising at least one transfer factor and at least one glucan, wherein said composition is selected from the group consisting of encapsulated transfer factor and glucan, time-release polymer wrapped transfer factor and glucan, and a combination thereof.

The following examples serve to more fully describe the manner of using the above-described invention. It is understood that these examples in no way serve to limit the true scope of this invention, but rather are presented for illustrative purposes.

### EXAMPLES

### Example 1

### Effects of Transfer Factor on Post-Vaccinal Antibody Titer in Swine

This study was undertaken to demonstrate the value of pure transfer factor derived from dairy colostrum and a formulation comprising transfer factor in titer enhancement. The vaccine tested was rabies vaccine IMRAB 3® (Lot number 12536b, available from Merial). Since pigs do not inherently carry a titer for rabies, the selected a study base of pigs would be expected to have immune systems naïve to this vaccine.

The study included 30 pigs, randomly selected 15 gilts (female) and 15 non-castrated male pigs ranging in age from 17 to 22 days. The animals were divided into three study groups of ten each, half female and half male. The pig groups were housed in individual pens after the study started.

Prior to the study, pigs were vaccinated at 10 days of age for mycoplasma, influenza, erysipelas and circo virus. All pigs are on the same pig starter diet (available from Sunglo) during this study.

On day 1 all pigs were bled, given 1 cc of Rabies vaccine subcutaneously and pigs were treated orally as described in TABLE 1, below. On day 1 and 21, serum was collected, and sent for testing at Kansas State Rabies Laboratory, Manhattan, Kans., where rabies titers were measured using a rapid fluorescent focus inhibition test.

TABLE 1
SWINE EXAMPLE-PROTOCOL

| | | Oral | Oral |
|---|---|---|---|
| | | administration | administration |
| | Oral | once daily, | once daily, |
| | administration | days 1 | days 9, 14 |
| PROTOCOL | once on day 1 | through 4 | and 19 |
| GROUP 1 | 6 cc tap water | 6 cc tap water | 6 cc tap water |
| (control) | | | |
| GROUP 2 | 1 gram pure | 1 gram pure | 1 gram pure |
| (pure transfer | transfer factor | transfer factor | transfer factor |
| factor derived | in 6 cc tap | in 6 cc tap | in 6 cc tap |
| from bovine | water | water | water |
| colostrum) | | | |
| GROUP 3 | 2 grams Form. A | 2 grams Form. A | 2 grams Form. A |
| (Formulation | in 6 cc tap | in 6 cc tap | in 6 cc tap |
| A-Table 1) | water | water | water |

Direct observations on day 21 of the study revealed that pigs treated with transfer factor in Groups 2 and 3 had the best appearance, even though three pigs from Group 3 started the study several pounds lighter or were younger pigs. Group 3 pigs, while still uneven in weight had the best coat and condition of all Groups. The control pigs lacked a show sheen or fullness of the transfer factor pigs from Groups 2 and 3.

The rapid fluorescent focus inhibition tests demonstrated increased titers in all Groups following initial titers <0.1 IU/ml, indicating that all pigs were naïve to rabies antigens at the beginning of the study.

A chart providing results of rabies titer measurements is provided in TABLE 2, below. The average titer for the 10 pigs in the control-Group 1 was 2.87 IU/ml. It is common for pigs not to mount a an immune response to rabies vaccines. Not surprisingly, three of the pigs in Group 1 did not mount an immune response. Remarkably, all the Group 2 pigs and 90% of the Group 3 pigs mounted an immune response. In Group 2, pigs treated with pure transfer factor averaged 26% higher titer than in Group 1, at 3.61 IU/ml. Group 3 pigs were treated with Formulation A (TABLE 3, 4 below) and mounted the highest average immune response, averaging 12.31 IU/ml. One pig in Group 3 did not mount a response.

TABLE 2
SWINE EXAMPLE-TITER RESULTS

| | Average | Average | | | | Oral |
|---|---|---|---|---|---|---|
| | rabies titer | rabies titer | Lowest | | Number of | administration |
| | prior to | 21 days | Highest | | pigs with | once daily, |
| | rabies | following | titer | | no increase | days 9, 14 |
| RESULTS | vaccination | vaccination | response | | titer | and 19 |
| GROUP 1 | <0.1 IU/ml | 2.87 IU/ml | <0.1 | IU/ml | 3 | 6 cc tap water |
| (control) | | | 11.0 | IU/ml | | |
| GROUP 2 | <0.1 IU/ml | 3.61 IU/ml | 0.5 | IU/ml | 0 | 1 gram pure |
| (pure transfer | | | 13.0 | IU/ml | | transfer factor |
| factor derived | | | | | | in 6 cc tap |
| from bovine | | | | | | water |
| colostrum) | | | | | | |
| GROUP 3 | <0.1 IU/ml | 5.55 IU/ml | <0.1 | IU/ml | 1 | 2 grams Form. A |
| (Formulation | | | >14.0 | IU/ml | | in 6 cc tap |
| A-Table 1) | | | | | | water |

TABLE 3
FORMULATION A
2000 mg per dose:

| COMPONENTS | DESCRIPTION | PERCENTAGE |
|---|---|---|
| Transfer factor | Mammalian colostrum* and | 38.4% |
| | avian blend including | |
| | avian antibodies | |
| Inositol | | 0.8% |
| hexaphosphate | | |
| Olive leaf | | 0.4% |
| extract | | |
| Ace mannans | | 0.4% |
| Poly r plus | cordyceps hybrid, | 10.5% |
| | cordyceps sinensis extract, | |
| | cordyceps millitaris, shitake, | |
| | maitake, innontus, obliquus, | |
| | poira cocos, coriolus agaricus | |
| | blazeii extract. | |
| Cordyceps sinensis | | 3.5% |
| extract | | |
| Agaricus blezei | cordyceps sinensis, | 4.4% |
| | grifola frondosa, | |
| | ganoderma lucidum, coriolus | |
| | versicolor lentinula edodes | |
| | blend 50% in the extract form | |
| | and 50% non in extract form | |
| Thymosine | | 4.4% |
| Vitamin E | | 5% |
| Zinc | | 1.7% |
| Electrolyte, | | 28% |
| vitamin blend | | |
| with probiotics | | |

*This colostrum blend indicated above contains higher molecular weight components including antibodies, proline-rich peptides, lactoferrin, growth factors in addition to the transfer factor filtrate.

TABLE 4
ELECTROLYTE, VITAMIN AND PROBIOTICS
IN FORMULATION A/SERVING OF 2000 MG

| Ingredient | Amount per 2000 mg serving | |
|---|---|---|
| Probiotics | 280 | mg |
| Dibasic potassium | 350 | mg |
| Citric acid | 95 | mg |
| Dextrose | 4032 | mg |
| Ascorbic acid | 7 | mg |
| Sodium chloride | 448 | mg |
| Magnesium sulfate | 22.4 | mg |
| Calcium pantothenate | 5.6 | mg |
| Vitamin A | 2.8 | mg |
| Vitamin D3 | .308 | mg |
| Riboflavin B2 | 3.58 | mg |
| Vitamin B12 | 39.20 | mg |
| Thiamine B1 | 3.36 | mg |
| Yeast extract | 56 | mg |
| Vitamin E | 2.46 | mg |
| Potassium chloride | 56 | mg |
| Glycine | 28 | mg |

This study would indicate that Formulation A and transfer factor alone had a positive effect on vaccine response of rabies vaccination in swine compared to control. There was notable improvement in condition and weight performance of the treated pigs.

TABLE 5
DATA SWINE RABIES TITERS

| | | Titer | | Titer |
|---|---|---|---|---|
| PatientID | DrawDate | IU/mL | DrawDate | IU/mL |
| 6C248 | Jun. 21, 2010 | <0.1 | Jul. 12, 2010 | 3.7 |
| 1C242 | Jun. 21, 2010 | <0.1 | Jul. 12, 2010 | 3.7 |
| 7C251 | Jun. 21, 2010 | <0.1 | Jul. 12, 2010 | <0.1 |
| 2C256 | Jun. 21, 2010 | <0.1 | Jul. 12, 2010 | <0.1 |
| 3C246 | Jun. 21, 2010 | <0.1 | Jul. 12, 2010 | 6.3 |
| 10C222 | Jun. 21, 2010 | <0.1 | Jul. 12, 2010 | 11 |
| 4C285 | Jun. 21, 2010 | <0.1 | Jul. 12, 2010 | 1 |
| 8C252 | Jun. 21, 2010 | <0.1 | Jul. 12, 2010 | <0.1 |
| 9C223 | Jun. 21, 2010 | <0.1 | Jul. 12, 2010 | 2.2 |
| 5C243 | Jun. 21, 2010 | <0.1 | Jul. 12, 2010 | 0.5 |
| 8RB237 | Jun. 21, 2010 | <0.1 | Jul. 12, 2010 | 78.6 |
| 1RB2410 | Jun. 21, 2010 | <0.1 | Jul. 12, 2010 | 1.6 |
| 4RB241 | Jun. 21, 2010 | <0.1 | Jul. 12, 2010 | 8 |
| 7RB291 | Jun. 21, 2010 | <0.1 | Jul. 12, 2010 | 11 |
| 5RB233 | Jun. 21, 2010 | <0.1 | Jul. 12, 2010 | 1.8 |
| 9RB234 | Jun. 21, 2010 | <0.1 | Jul. 12, 2010 | 2.6 |
| 10RB232 | Jun. 21, 2010 | <0.1 | Jul. 12, 2010 | 2.6 |
| 2RB2510 | Jun. 21, 2010 | <0.1 | Jul. 12, 2010 | <0.1 |
| 3RB281 | Jun. 21, 2010 | <0.1 | Jul. 12, 2010 | 4.8 |
| 6RB292 | Jun. 21, 2010 | <0.1 | Jul. 12, 2010 | 12 |
| 2TF258 | Jun. 21, 2010 | <0.1 | Jul. 12, 2010 | 0.7 |
| 9TF221 | Jun. 21, 2010 | <0.1 | Jul. 12, 2010 | 2.6 |
| 1TF224 | Jun. 21, 2010 | <0.1 | Jul. 12, 2010 | 0.5 |
| 3TF283 | Jun. 21, 2010 | <0.1 | Jul. 12, 2010 | 2.6 |
| 8TF244 | Jun. 21, 2010 | <0.1 | Jul. 12, 2010 | 4.3 |
| 7TF247 | Jun. 21, 2010 | <0.1 | Jul. 12, 2010 | 4 |
| 5TF245 | Jun. 21, 2010 | <0.1 | Jul. 12, 2010 | 13 |
| 6TF253 | Jun. 21, 2010 | <0.1 | Jul. 12, 2010 | 0.5 |
| 10TF282 | Jun. 21, 2010 | <0.1 | Jul. 12, 2010 | 7 |
| 4TF231 | Jun. 21, 2010 | <0.1 | Jul. 12, 2010 | 0.9 |

### Example 2

### Effect of Parenteral Time-Release Transfer Factor on Rabies Titer Enhancement in Goats

The effect of parenterally administered transfer factor and glucans was studied in a pair of twin goat bucklings. One of the twins was administered a time-release pelleted transfer factor and a time-release pelleted hybrid cordyceps at the same time as the vaccination for rabies. The pellets containing transfer factor and glucans were injected on the second twin served as the control, receiving only rabies vaccination. 21 days after vaccination, the treated goat had a four-fold increase in rabies titer compared with the control.

Blood was taken from both 80 pound goats #227 (control) and #228 (treated) on day 1 followed by vaccination with 1 cc of IMRAB 3® subcutaneously and the following treatment in goat #228. 424 mg of active transfer factor wrapped with the time-release composition described in TABLE 6, below, was administered subcutaneously to goat #228 in eight pellets on the right side. 424 mg of actives comprising cordyceps sinensis hybrid wrapped with the time-release composition described in TABLE 6 was administered subcutaneously to goat #228 in eight pellets on the left side at the same time. No placebo was administered to goat #227. Feeding and housing of goats 227 and 228 were identical.

TABLE 6
PURE TRANSFER FACTOR CONTROLLED-
RELEASE FORMULATION

| Ingredient | Percent by weight | |
|---|---|---|
| “Active” powder blend | 67.5% | w/w |
| Hydroxypropyl cellulose (controlled release | 30.0% | w/w |
| excipient) | | |
| Calcium stearate (pelleting lubricant) | 2.0% | w/w |
| Fumed silica (powder glidant) | 0.5% | w/w |

Blood samples taken day one and 21 were tested for rabies titer using rapid fluorescent focus inhibition test. Titers increased from <1:5 in control goat #227 to 1:340; and from <1:5 to 1:1200 in treated goat #228. Results indicate an approximately 353% increase or a four-fold increase in rabies titer response for the study goat treated with time-release transfer factor and glucan extract.

### Example 3

### Effects of Transfer Factor on Vaccine Titers in Feedlot Cattle

44 head of stockers, 500 pound feedlot cattle (approx. 200 days old) were tested for swine parvovirus and leptospirosis titers, following vaccination using vaccine manufactured for use in swine. The cattle were divided into 3 groups: one control group of 5 cattle and 20 cattle in Groups 2 and 19 cattle in Group 3, which were fed either immediate release oral transfer factor (see TABLE 7, below) or a combination of immediate and time-release oral transfer factor compositions (see TABLES 9, 10, 11, below). Following routine processing described in TABLE 8 the protocol described in TABLE 7 below, was utilized.

TABLE 7
FEEDLOT PROTOCOL

| | # in | | Days of |
|---|---|---|---|
| PROTOCOL | group | Oral treatment | treatment |
| Group 1 (Control) | 5 | | |
| Group 2 immediate | 20 | One gelatin capsule | Day 1, 2 |
| release formula | | containing 0.83 oz | and 12 |
| | | of TABLE 4 formula | |
| Group 3 | 19 | One gelatin capsule | Day 1 |
| immediate release | | containing 0.83 oz | and 12 |
| formula plus time- | | of TABLE 4 and two | |
| release formula | | gelatin capsules of | |

TABLE 6 & 7 formula

TABLE 8
PROCESSING OF CATTLE PRIOR TO EXPERIMENT

| DATE | PROCEDURE |
|---|---|
| Oct. 7, | Cattle Delivered |
| 2009 | |
| | Tagged and grouped |
| | All vaccinated for: Infectious Bovine Rhinotracheitis |
| | (IBR), Parainfluenza virus (PI3), Bovine Viral |
| | Diarrhea (BVD), Bovine Respiratory Syncytial Virus |
| | (BRSV)* |
| | ST-BAC** implant, dewormed, treated for ectoparasites |

*Note:
100% of the cattle were subsequently diagnosed with positive titers indicating BRSV infection; 40% infection with BVD and 40% infection with PI3.
**vaccine for Histophilus somni, Mannheimia, Haemolytica, Mycoplasma bovis, Pasteurella multocida

TABLE 9
TRANSFER FACTOR LIVESTOCK STRESS RUMEN BY-PASS FORMULATION**
Amounts in mg/lb of body weight unless otherwise stated Stabilized components*

| | Amount: mg/oz | |
|---|---|---|
| | (unless otherwise | |
| Ingredient | noted) of formula | |
| Transfer factor (mammal source)* | 3500.0 | |
| Transfer factor (avian source)* | 1000.0 | |
| β-sitosterol (90% phytosterols)* | 300.0 | |
| Inositol hexaphosphate* | 350.0 | |
| Olive leaf extracts* | 35.0 | |
| Aloe extract powder* (200:1) | 17.0 | |
| Hybridized and non-hybridized Glucans* | 4000.0 | |
| (from Hybridized Cordyceps sinensis, | | |
| Agaricus blazeii, Miatake, Shitake, | | |
| Coriolis, Inonotus, Obliquus, and | | |
| Poris cocos mushrooms) | | |
| Vitamin C* | 2000.0 | |
| Vitamin A | 4434 | IU/oz |
| Vitamin D3 | 1140 | IU/oz |
| Vitamin E | 500 | IU/oz |
| Vitamin B1 | 12.77 | |
| Vitamin B2 | 12.77 | |
| Vitamin B12 | 1.5 | |
| Di-potassium phosphate | 1.5 | g/oz |
| Potassium chloride | 207 | |
| Magnesium sulfate | 83 | |
| Calcium pantothenate | 23 | |
| Ascorbic acid | 23 | |
| Lactic acid bacteria | 2.5 × 10⁶ | CFU/oz |
| Yeast (S. cerevisiae) | 15.0 × 10⁶ | CFU/oz |

| | |
|---|---|
| Zinc proteinate | 10 |

*Stabilized active ingredients are included in a formulation of 50% soybean oil and 50% active ingredient.
**These amounts are calculated for livestock animals weighing about 450 to 1,000 pounds, goats weighing about 150 pounds, and dogs and cats weighing from about 8 to about 15 pounds.

TABLE 10
ACTIVE BLEND (PER SERVING)

| INGREDIENT | AMOUNT | |
|---|---|---|
| Mammalian transfer factor blend (transfer factor, | 5103 | mg |
| antibodies, high molecular weight colostral | | |
| fraction) | | |
| Avian transfer factor | 773 | mg |
| IP6, olive leaf extract, acemannan, phytosterol | 644 | mg |
| PolyR | 3289 | mg |
| Porcine thymosin | 1610 | mg |

TABLE 11
TIME-RELEASE FORMULATION
Once Active blend is coated with formulation below, the composition is
formed into pellets. These pellets are placed inside gelatin capsules
(size 7) for oral administration.

| | |
|---|---|
| INGREDIENT (Function) | AMOUNT |

| | | |
|---|---|---|
| Active powder blend from Table 6 | 50% | w/w |
| Ethylcellulose (filler and controlled release | 39.5% | w/w |
| polymer) | | |
| Hydroxypropyl cellulose (binder and pore-former) | 8.0% | w/w |
| Stearic acid (pelleting lubricant) | 2.0% | w/w |
| Fumed silica (Powder glidants) | 0.5% | w/w |

Cattle in Groups 2 and 3 showed three times the rate gain as the controls, and had a 220% increase in titer to both swine parvovirus and leptospirosis. 100% of the control group had no response to leptospirosis vaccination at day 21, while 90% from Group 2 and 89% from Group 3 had titers at day 21, which remained stable or increased by day 42 in 61% of Group 2 and 65% of the Group 3 responders as demonstrated in Table 12-, below. Response to parvovirus vaccine was equally unexpected in the treated Groups as compared to the controls. Group 2 and 3 mounted a 4.5 and 5 times greater immune antibody titer response to the leptospirosis vaccine than controls, respectively; and a 3.2 and 4.6 times greater titer response to parvo virus vaccine than controls. Notably, there was significantly slower vaccine decay in both treated groups compared with controls.

In addition to the direct immune modulating response, the Group 2 and 3 cattle averaged 3.17 and 2.78 pounds of weight gain per day by day 42 compared with only 1.51 pounds of weight gain per day in the control group. Notably, there were no side effects in the treated cattle groups to the swine vaccines.

TABLE 12
SUMMARY OF COMPARED TITER RESPONSE
IMMUNO MODULATION TO INCREASE ANTIBODY TITER RESPONSE

| | C-LIT | RB-LIT | RBTF-LIT |
|---|---|---|---|
| 0 DAY | <100/neg | <100/neg | <100/neg |
| 21 D | <100/neg | 200.0:1 | 263.0:1 |
| 42 D | <100/neg | 190.0:1 | 174.0:1 |
| | C-PVT | RB-PVT | RBTF-PVT |
| 0 DAY | 46/neg | <16/neg | <16/neg |
| 21 D | 80.0:1 | 259.0:1 | 368.0:1 |
| 42 D | 2.2:1 | 239.0:1 | 273.0:1 |

CONTROL = C (N = 5)
RUMEN BY-PASS = RB (N = 19)
RUMEN BY-PASS -TIME RELEASE = RBTF (N = 20)
LEPTO ICTERO TITRE = LIT
PARVO VIRUS TITRE = PVT
*titres are mean values of the group.

TABLE 15
CONTROL GROUP DATA
CONTROLS

| | 0 DAY | 21 D | 42 D | | | 0 D | 21 D | 42 D | | 0 D | 21 D | 41 D |
|---|---|---|---|---|---|---|---|---|---|---|---|---|
| TAG | WT | WT | WT | #GAINED | ADG | LEP | LEP | LEP | ICTERO | PPV | PPV | PPV |
| 9-056 | 592 | 656 | | | 3.04 | <100/NEG | <100/NEG | | | <16/NEG | 64/* | |
| 9-058 | 586 | 650 | | | 3.01 | <100/NEG | <100/NEG | | | 32/* | 256/* | |
| 9-057 | 532 | 548 | | | 0.76 | <100/NEG | <100/NEG | | | <16/NEG | 64/* | |
| 9-052 | 477 | 453 | | | LOSS 24 # | <100/NEG | <100/NEG | | | 16/* | 32/* | |
| 9-049 | 474 | 499 | | | 1.19 | <100/NEG | <100/NEG | | | <16/NEG | 64/* | |
| | | AV: | | | | | AV. 1.6 | | | | AV2: 1 | |
| | | 29# | | | | | | | | | | |

TABLE 16
GROUP 2 DATA
TREATS RB

| | 0 DAY | 21 D | 42 D | | | 0 D | 21 D | 42 D | | 0 DA | 21 D | 42D |
|---|---|---|---|---|---|---|---|---|---|---|---|---|
| TAG | WT | WT | WT | # GAINED | ADG | LEP | LEP | LEP | ICTERO | PPV | PPV | PPV |
| 9-001 | 436 | 520 | 622 | 186 | 4.42 | <100/NEG | 100/* | 100/* | | 32/* | 512/* | 32/* |
| 9-011 | 494 | 596 | 620 | 126 | 3 | <100/NEG | 400/* | 200/* | | <16/NEG | 16/* | 200/* |
| 9-053 | 460 | 564 | 650 | 190 | 4.52 | <100/NEG | 200/* | 200/* | | 16/* | 256/* | 256/* |
| 9-015 | 411 | 457 | 484 | 73 | 1.74 | <100/NEG | <100/NEG | <100/NEG | | 32/* | 16/* | 64/* |
| 9-017 | 452 | 499 | 560 | 108 | 2.57 | <100/NEG | 200/* | 200/* | | 16/* | 256/* | 256/* |
| 9-019 | 473 | 508 | 586 | 113 | 2.69 | <100/NEG | 400/* | 800/* | | <16/NEG | 128/* | 256/* |
| 9-051 | 365 | 437 | 452 | 87 | 2.07 | <100/NEG | 100/* | 100/* | | <16/NEG | 64/* | 100/* |
| 8-023 | 398 | 397 | 432 | 34 | 0.81 | <100/NEG | 100/* | 100/* | | <16/NEG | 512/* | 100/* |
| 9-055 | 449 | 554 | 670 | 221 | 5.26 | <100/NEG | 400/* | 200/* | | <16/NEG | 256/* | 512/* |
| 9-061 | 432 | 499 | 550 | 118 | 2.8 | 100/* | 200/* | 100/* | | 128/* | 256/* | 64/* |
| 9-029 | 340 | 394 | 497 | 157 | 3.74 | <100/NEG | 200/* | 100/* | | 16/* | 32/* | 100/* |
| 9-031 | 385 | 470 | 522 | 137 | 3.26 | <100/NEG | 100/* | <100/NEG | | <16/NEG | 128/* | 64/* |
| 9-033 | 423 | 499 | 552 | 120 | 2.86 | <100/NEG | 100/* | 100/* | | <16/NEG | 512/* | 256/* |
| 9-035 | 407 | 530 | 594 | 187 | 4.45 | <100/NEG | 100/* | 200/* | | 32/* | 128/* | 256/* |
| 9-037 | 346 | 421 | 508 | 162 | 3.86 | <100/NEG | 400/* | 100/* | | 32/* | 256/* | 512/* |
| 9-039 | 351 | 384 | 397 | 46 | 1.09 | <100/NEG | 200/* | 100/* | | <16/NEG | 1024/* | 512/* |
| 9-041 | 406 | 492 | 532 | 126 | 3 | <100/NEG | 200/* | 800/* | | <16/NEG | 256/* | 512/* |
| 9-043 | 424 | 517 | 602 | 178 | 4.24 | <100/NEG | 200/* | 200/* | | <16/NEG | 256/* | 512/* |
| 9-045 | 428 | 552 | 614 | 186 | 4.43 | <100/NEG | 100/* | 200/* | | <16/NEG | 256 | 200/* |
| 9-047 | 375 | 417 | 481 | 106 | 2.53 | <100/NEG | <100/NEG | <100/NEG | | <16/NEG | 32/* | 16/* |
| | AV: | AV: | | AV: | ADG: | | | | | | | |
| | 413# | 72.5# | | 133# | 3.17# | | | | | | | |
| | | ADG: | | | | | | | | | | |
| | | 3.45# | | | | | | | | | | |

TABLE 17
GROUP 3 DATA
TREATS RB-TR

| | 0 DAY | 21 D | 42 D | | | 0 D | 21 D | 42 D | | 0 DA | 21 D | 42 D |
|---|---|---|---|---|---|---|---|---|---|---|---|---|
| TAG | WT. | WT | WT | # GAINED | ADG | LEP | LEP | LEP | ICTERO | PPV | PPV | PPV |
| 9-054 | 423 | 522 | 542 | 119 | 2.83 | <100/NEG | 800/* | 400/* | | 32/* | 128/* | <16/NEG |
| 9-012 | 327 | 402 | 414 | 87 | 2.07 | <100NEG | 200/* | 400/* | | 32/* | 128/* | 400/* |
| 9-014 | 469 | 576 | 652 | 183 | 4.35 | <100/NEG | 200/* | 200/* | | 16/* | 256/* | 64/* |
| 9-016 | 458 | 552 | 598 | 140 | 3.3 | <100/NEG | 100/* | 400/* | | 16/* | 256/* | 128/* |
| 9-018 | 445 | 498 | 516 | 71 | 1.69 | <100/NEG | <100/NEG | <100/NEG | | 16/* | 64/* | 64/* |
| 9-020 | 335 | 391 | 424 | 89 | 2.11 | <100/NEG | 200/* | 100/* | | <16/NEG | 256/* | 256/* |
| 9-024 | 329 | 383 | 423 | 94 | 2.23 | <100/NEG | 200/* | 200/* | | 16/* | 256/* | 64/* |
| 9-026 | 395 | 510 | 568 | 173 | 4.11 | <100/NEG | 100/* | 100/* | | 2048/* | 2048/* | 2048/* |
| 9-028 | 450 | 560 | 610 | 160 | 3.81 | <100/NEG | 800/* | 100/* | | 16/* | 32/* | 32/* |
| 9-030 | 388 | 418 | 436 | 48 | 1.14 | <100/NEG | 400/* | 100/* | | <16/NEG | 512/* | 512/* |
| 9-032 | 386 | 456 | 546 | 160 | 3.81 | <100/NEG | <100/NEG | <100/NEG | | 32/* | 128/* | 128/* |
| 9-034 | 427 | 467 | 476 | 49 | 1.16 | <100/NEG | 200/* | 100/* | | <16NEG | 512/* | 100/* |
| 9-036 | 466 | 574 | 614 | 148 | 3.52 | <100/NEG | 100/* | 100/* | | <16/NEG | 256/* | 256/* |
| 9-038 | 431 | 538 | 622 | 191 | 4.54 | <100/NEG | 400/* | 200/* | | 32/* | 512/* | 16/* |
| 9-050 | 320 | 414 | 495 | 175 | 4.16 | <100/NEG | 100/* | 100/* | | <16/NEG | 1024/* | 512/* |
| 9-066 | 449 | 546 | 606 | 157 | 3.74 | 800/* | 800/* | 200/* | | <16NEG | 32/* | 200/* |
| 9-044 | 423 | 546 | 620 | 197 | 4.69 | <100/NEG | 100/* | 200/* | | <16/NEG | 32/* | 200/* |
| 9-046 | 380 | 434 | 453 | 73 | 1.73 | <100/NEG | 100/* | 200/* | | <16/NEG | 512/* | 200/* |
| 9-048 | 413 | 532 | 586 | 173 | 4.11 | <100/NEG | 200/* | 100/* | | <16/NEG | 64/* | 16/* |
| | | AV: | | AV: | ADG: | | | | | | | |
| | | 82# | | 117# | 2.78# | | | | | | | |
| | | ADG: | | | | | | | | | | |
| | | 3.92# | | | | | | | | | | |

### Example 4

### Effect of Transfer Factor Administer Orally to Cats During and Following Rabies Vaccination

A cat 20 years old, when titer for rabies was attempted, was essentially negative for two titers (e.g., titers of approximately 0.17 IU/mL), having been given rabies vaccination three consecutive years. After administration of the transfer factor formulation (see TABLE 18, below) one tsp daily in drinking water for several weeks, and 0.15 cc of rabies vaccine or 15% of a standard dose, the rabies titer was raised to approximately 1.52 IU/mL.

A 19 week old kitten was administered rabies vaccination, 1 cc IMRAB 3® subcutaneously following a test for rabies titer revealing a titer of <1 UL/ml, considered a negative titer. The kitten was fed 1500 mg. transfer factor in a chew formulation daily for 42 days. 21 days later the kittens rabies titer was greater than 14 IU/ml and by day 42 it increased to 15 IU/ml. The administration of transfer factor with and following vaccination unexpectedly boosts the immune response for a prolonged time period.

TABLE 18
TRANSFER FACTOR FORMULATION (NO ENCAPSULATION)
(Amounts in mg/lb of body weight unless otherwise stated)

| | | | | Dosage: |
|---|---|---|---|---|
| | | | | mg/oz of |
| Component | High | Low | Preferred | formula |
| Calcium Pantothenate | 1.80 | 0.09 | 0.028 | 28.00 |
| Vitamin C (ascorbic | 20.00 | 0.056 | 0.017 | 17.00 |
| acid) | | | | |
| Vitamin B₁₂ | 13.00 | 0.13 | 0.198 | 198.59 |
| Vitamin A | 600.00 IU | 0.10 IU | 0.014 | 14.00 |
| Vitamin B₂ | 1.20 | 0.065 | 0.018 | 18.00 |
| Thiamine | 16.00 | 0.0308 | 0.017 | 17.00 |
| Vitamin E | 72.9 IU | 0.729 IU | 0.012 | 12.48 |
| Magnesium Sulfate | 10.00 | 0.113 | 0.113 | 113.00 |
| *Lactobacillus | 10.00 | 0.467 | 1.418 | 1418.00 |
| acidophilus | | | | |
| (10⁹ colony forming | | | | |
| units (CFU)/gm) | | | | |
| Sodium Chloride | 166.00 | 0.236 | 2.368 | 2368.00 |
| Dipotassium phosphate | 116.00 | 5.85 | 1.773 | 1773.00 |
| Citric acid | 31.00 | 1.59 | 0.482 | 482.00 |
| Yeast (hydrolyzed) | 180.00 | 0.1957 | 0.283 | 283.00 |
| Glycine | 0.142 | 0.0142 | 0.142 | 141.80 |
| Potassium chloride | 18.00 | 0.93 | 0.283 | 283.00 |
| Vitamin D3 | 29.00 | 0.729 | 0.002 | 1.56 |
| Dextrose | 40.00 | 2.00 | 21.38 | 21375.00 |
| Artificial flavor | 0.028 | 0.0028 | 28.548 | 28.30 |
| Transfer Factor | 50.00 | 0.05 | 0.75 | 750.00 |
| Sipernat (silicon dioxide) | | | 0.05 | 56.70 |

It will be apparent to those skilled in the art that various modifications and variations can be made in the methods and compositions of the present inventions without departing from the spirit or scope of the invention. Thus, it is intended that the present invention cover the modification and variations of the inventions provided they come within the scope of the appended claims and their equivalents.

The terms and expressions which have been employed are used as terms of descriptions and not of limitation, and there is no intention that in the use of such terms and expressions of excluding any equivalents of the features shown and described or portions thereof, but it is recognized that various modifications are possible within the scope of the invention. Thus, it should be understood that although the present invention has been illustrated by specific embodiments and optional features, modification and/or variation of the concepts herein disclosed may be resorted to by those skilled in the art, and that such modifications and variations are considered to be within the scope on this invention.

In addition, where features or aspects of the invention are described in terms of Markush group or other grouping of alternatives, those skilled in the art will recognized that the invention is also thereby described in terms of any individual member or subgroup of members of the Markush group or other group.

Unless indicated to the contrary, all numerical ranges described herein include all combinations and subcombinations of ranges and specific integers encompassed therein. Such ranges are also within the scope of the described invention.

The disclosures of each patent, patent application and publication cited or described in this document are hereby incorporated herein by reference, in their entirety.

1. A method of improving a vaccine-induced immune response in a subject, comprising the step of administering to a subject in need thereof an effective amount of a composition comprising at least one transfer factor selected from the group consisting of bovine colostrum transfer factor, avian transfer factor, and a combination thereof.

2. The method of claim 1 wherein the subject is avian or a mammal.

3. The method of claim 2 wherein the mammal is selected from the group consisting of human and non-human primates, cattle, horses, swine, sheep, goats, deer, lama, dogs and cats.

4. The method of claim 1 wherein the composition is administered orally or parenterally.

5. The method of claim 1 wherein the composition further comprises at least one glucan.

6. The method of claim 1 wherein at least one transfer factor is encapsulated with a hydrophobic coating that is between about 25 and about 150 wt % of the transfer factor.

7. The method of claim 1 wherein the composition comprises a time-released coating comprising a combination of water soluble and water insoluble cellulose polymer.

8. A kit comprising at least a first container comprising a composition comprising at least one transfer factor, wherein said composition is selected from the group consisting of encapsulated transfer factor, time-release polymer wrapped transfer factor, and a combination thereof.

9. A kit comprising at least a first container comprising a composition comprising at least one transfer factor and at least one glucan, wherein said composition is selected from the group consisting of encapsulated transfer factor and glucan, time-release polymer wrapped transfer factor and glucan, and a combination thereof.

10. The method of claim 5 wherein the composition comprises as time-released coating comprising a combination of water soluble and water insoluble cellulose polymer.

11. The method of claim 6 wherein the composition comprises a time-released coating comprising a combination of water soluble and water insoluble cellulose polymer.
